(19) United States
(12) Patent Application Publication
(10) Pub. No.: US 2020/0191784 A1
(43) Pub. Date: Jun. 18, 2020

(54) BIOMARKER COMPOSITION FOR DIAGNOSIS OF SYSTEMIC LUPUS ERYTHEMATOSUS COMPRISING AIMP1 AND METHOD FOR DIAGNOSING SYSTEMIC LUPUS ERYTHEMATOSUS USING SAME

(71) Applicants: AJOU UNIVERSITY INDUSTRY-ACADEMIC COOPERATION FOUDATION, Suwon-si, Gyeonggi-do (KR); INDUSTRY-ACADEMIC COOPERATION FOUNDATION, YONSEI UNIVERSITY, Seodaemun-gu (KR)

(72) Inventors: Sang Gyu PARK, Seoul (KR); Sang Won LEE, Seoul (KR)

(73) Assignees: AJOU UNIVERSITY INDUSTRY-ACADEMIC COOPERATION FOUDATION, Suwon-si Gyeonggi-do (KR); INDUSTRY-ACADEMIC COOPERATION FOUNDATION, YONSEI UNIVERSITY, Seodaemun-gu (KR)

(21) Appl. No.: 16/608,931

(22) PCT Filed: Apr. 30, 2018
(86) PCT No.: PCT/KR2018/005001

§ 371 Date: Oct. 28, 2019

(30) Foreign Application Priority Data
Apr. 28, 2017 (KR) 10-2017-0055247

(51) Int. Cl. G01N 33/564

(52) U.S. Cl. CPC G01N 33/564; G01N 2800/52; G01N 2800/104

(57) ABSTRACT

The present invention relates to a biomarker composition for diagnosing SLE comprising an AIMP1 as an active ingredient and a method of diagnosing SLE using the same, and particularly the present invention provides a biomarker composition for diagnosing SLE comprising an AIMP1, a kit for diagnosing SLE and a method of diagnosing SLE using the same. Also, the present invention provides a biomarker composition for predicting SLE prognosis comprising AIMP1 as an active ingredient, a kit for predicting SLE prognosis and a method for predicting SLE prognosis using the same. Therefore, AIMP1 of the present invention can be usefully used for diagnosing SLE and predicting SLE prognosis.

### TECHNICAL FIELD

The present invention relates to a biomarker composition for diagnosing systemic lupus erythematosus (SLE) comprising an aminoacyl-tRNA synthetase complex interacting multifunctional protein-1 (AIMP1) and a method of diagnosing systemic lupus erythematosus using the same.

### BACKGROUND ART

Systemic lupus erythematosus (SLE) is a prototypicalsystemic autoimmunedisease, which is characterized by excessive autoantibody production and immune complex formation in its pathophysiology. So far, various autoreactive immune cells have been discovered to contribute to the development and exacerbation of SLE. Among them, dendritic cells and B cells still remain at the forefront of the pathogenesis of SLE, and they can promote the production of interferon-α as well as autoantibodies. Also, autoreactive immune cells can activate nuclear factor kappa B (NF-κB) via extracellular signal-regulated kinase (ERK)/mitogen-activated protein kinase (MAPK) pathway. Activated NF-κB, in turn, promote the expression of its downstream genes including interferon-γ, IL-1, IL-2, IL-6, IL-12, IL-17 and tumour necrosis factor (TNF)-α. In addition, SLE may be affected by the alteration in T cell population such as decreased Treg cells and increased Th17 cells and follicular helper T cells. Therefore, if there be a molecule to reflect dysregulation of autoreactive immune cells and imbalance of cytokines in the blood of SLE patients, it must be a good biomarker to predict disease activity of SLE.

Transfer Ribonucleic acids (tRNAs) generally consist of 75-95 nucleotides, and tRNAs play pivotal important roles in the process of protein translation by carrying specific amino acids to the ribosomes based on individualized codons of messenger RNA. So far, 20 different types of tRNAs have been discovered in humans, and each amino acid is charged with a cognate tRNA via aminoacyl-tRNA synthetases (ARSs). Compared to prokaryotic ARSs, ARS in mammals forms a multi-tRNA synthetase complex including 11 different ARSs and 3 non-enzymatic factors such as aminoacyl-tRNA synthetase-interacting multifunctional protein (AIMP)1/p43, AIMP2/p38 and AIMP3/p18. AIMPs appear to participate in the assembly of the complex-forming enzymes with evidences as follows: 1) AIMPs are tightly linked with each other, resulting in the interplay of the intracellular stability of each AIMP; 2) each AIMP has its preferably interacting enzymes; 3) AIMP1, especially, seems to be a crucial cofactor due to its centric localization in the multi-tRNA synthetase complex, which suggests that it may facilitate the delivery of tRNA to the catalytic sites of bound ARSs.

Apart from the function of AIMP1 bound to the multi-tRNA synthetase complex, AIMP1 could be secreted to the circulation on hypoxia and both apoptotic/necrotic cell death and it may have several immune-stimulatory effects: first, secretory AIMP1 may promote angiogenesis by ERKs activation; second, AIMP1 may stimulate monocytes and macrophages to produce pro-inflammatory cytokines such as TNF-α, interleukin (IL)-6, IL-8 and macrophage chemotactic protein (MCP)-1 via p38 MAPK and NF-κB; third, AIMP1 may induce dendritic cell maturation and increase IL-6 and IL-12 production. In addition, we had previously demonstrated that serum AIMP1 was measured in rheumatoid arthritis patients higher than healthy controls and monoclonal antibody targeting AIMP1 significantly ameliorated the severity of arthritis and reduced serum IL-1β, IL-8, MCP-1 and TNF-α in mice with collagen induced arthritis. However, the relationship between AIMP1 and SLE has not been confirmed.

### DISCLOSURE

### Technical Problem

It is an object of the present invention to provide a biomarker composition for diagnosing SLE or for predicting SLE prognosis comprising AIMP1 as an active ingredient, and a method of diagnosing SLE or predicting SLE prognosis using the same.

### Technical Solution

The present invention provides a biomarker composition for diagnosing SLE comprising AIMP1 as an active ingredient.

The present invention also provides a composition for diagnosing SLE comprising an agent capable of measuring a level of AIMP1 as an active ingredient.

In addition, the present invention provides a kit for diagnosing SLE comprising the composition for diagnosing SLE.

In addition, the present invention comprises a method of providing information necessary for diagnosing SLE comprising: (1) measuring AIMP1 level from samples isolated from SLE patients; (2) comparing measured AIMP1 level with a control sample; and (3) determining as SLE when the measured AIMP1 level is higher than that of the control sample.

In addition, the present invention provides a method of providing information necessary for diagnosing SLE comprising: (1) measuring AIMP1 level from samples isolated from SLE patients; and (2) determining as SLE when a measured AIMP1 level is 5 to 20 ng/mL.

Furthermore, the present invention provides a biomarker composition for predicting SLE prognosis comprising AIMP1 as an active ingredient.

In addition, the present invention provides a composition for predicting SLE prognosis comprising an agent capable of measuring the level of AIMP1 as an active ingredient.

The present invention also provides a kit for predicting SLE prognosis comprising the composition for predicting SLE prognosis.

In addition, the present invention provides a method of providing information necessary for predicting SLE prognosis comprising: (1) measuring AIMP1 level from samples isolated from SLE patients; and (2) determining as active SLE when a measured AIMP1 level is 10 to 20 ng/mL.

### Advantageous Effects

The present invention relates to a biomarker composition for diagnosing SLE comprising an AIMP1 as an active ingredient and a method of diagnosing SLE using the same and particularly the present invention provides a biomarker composition for diagnosing SLE comprising an AIMP1, a kit for diagnosing SLE and a method of diagnosing SLE using the same. Also, the present invention provides a biomarker composition for predicting SLE prognosis comprising AIMP1 as an active ingredient, a kit for predicting SLE prognosis and a method for predicting SLE prognosis using the same. Therefore, AIMP1 of the present invention can be usefully used for diagnosing SLE and predicting SLE prognosis.

### DESCRIPTION OF DRAWINGS

FIG. 1 shows the comparison of serum AIMP1 between patients with SLE and healthy controls. Both patients with active and stable SLE showed the higher median serum AIMP1 than healthy controls. Data are expressed as median and the error bars indicate interquartile ranges (IQR). *p<0.001.

FIG. 2 shows the correlation of serum AIMP1 with laboratory variables related to either disease activity or inflammatory burdens in patients with SLE. Serum AIMP1 was significantly correlated with SLEDAI-2K and furthermore, serum AIMP1 was also correlated with laboratory variables related to either disease activity or inflammatory burdens.

FIG. 3 shows the optimal cut off of serum AIMP1 to predict active SLE. Patients with serum AIMP1≥10.09 ng/mL had active SLE more frequently than those with serum AIMP1<10.09 ng/mL [(80.5% (29/36 patients) vs 49.1% (61/124 patients)].

### BEST MODE

Accordingly, the present inventors confirmed the association between serum AIMP1 and SLE disease activity under the assumption that the onset of SLE and secretory AIMP1 may be related to each other, and predicted the active SLE based on the SLE disease activity index (SLEDAI)-2K, thereby completing the present invention.

The present invention provides a biomarker composition for diagnosing systemic lupus erythematosus (SLE) comprising an aminoacyl-tRNA synthetase complex interacting multifunctional protein-1 (AIMP1) as an active ingredient.

As used herein, the term “diagnosis” refers to determining the susceptibility of an object to a particular disease or disorder, determining whether an object currently has a particular disease or disorder, determining the prognosis of a subject with a particular disease or disorder, or therametrics (eg, monitoring the condition of the object to provide the information regarding treatment efficacy).

Also, the present invention provides a composition for diagnosing SLE comprising an agent capable of measuring the level of AIMP1 as an active ingredient.

The agent for measuring the level of AIMP1 can be included without limitation as long as it can be performed by a method known in the art, for example, may include antibody, peptide, aptamer or compound that specifically binds to the AIMP1, but it is not limited thereto.

In addition, the present invention provides a kit for diagnosing SLE comprising the composition for diagnosing SLE.

In addition, the present invention comprises a method of providing information necessary for diagnosing SLE comprising: (1) measuring AIMP1 level from samples isolated from SLE patients; (2) comparing measured AIMP1 level with a control sample; and (3) determining as SLE when the measured AIMP1 level is higher than that of the control sample.

In addition, the present invention provides a method of providing information necessary for diagnosing SLE comprising: (1) measuring AIMP1 level from samples isolated from SLE patients; and (2) determining as SLE when a measured AIMP1 level is 5 to 20 ng/mL.

As used herein, the term “sample” includes samples such as tissue, cells, blood, serum, plasma, saliva, sputum, cerebrospinal fluid, or urine that differs from the control in AIMP1 levels, but it is not limited thereto. Preferably it may be blood, more preferably serum.

The present invention also provides a biomarker composition for predicting SLE prognosis comprising AIMP1 as an active ingredient.

As used herein, the terms “marker”, “biological marker”, “biomarker” are used interchangeably. The marker is generally a detectable molecule or compound in a biological sample, and refers to an indicator capable of detecting a specific change in a living body. In the present invention, the marker is AIMP1, and their metabolites are also included in the scope of the present invention.

SLE can be diagnosed or the prognosis can be predicted by measuring their levels.

In addition, the present invention provides a composition for predicting SLE prognosis comprising an agent capable of measuring the level of AIMP1 as an active ingredient.

The agent for measuring the level of AIMP1 can be included without limitation as long as it can be performed by a method known in the art, for example, may include antibody, peptide, aptamer or compound that specifically binds to the AIMP1, but it is not limited thereto.

Furthermore, the present invention provides a kit for predicting SLE prognosis comprising the composition for predicting SLE prognosis.

As used herein, the term “antibody” refers to a specific immunoglobulin directed to an antigenic site as a term known in the art. All those prepared by injecting at least one of the above mentioned proteins, or those sold commercially, is available. In addition, the antibodies include polyclonal antibodies, monoclonal antibodies, fragments capable of binding epitopes, and the like. Forms of such antibodies include polyclonal antibodies or monoclonal antibodies, including all immunoglobulin antibodies. The antibody means a complete form having two light chains of full length and two heavy chains of full length. In addition, the antibody also contains special antibodies such as a humanized antibody, etc.

In addition, the kit of the present invention includes an antibody that specifically binds to a marker component, a secondary antibody conjugate in which a label developed by reaction with a substrate is conjugated, a color substrate solution to be color-reacted with the label, a wash solution, and an enzyme stopping solution and the like, and may be prepared as a number of separate packaging or compartments containing the reagent components used.

As used herein, the term “peptide” has the advantage of high binding capacity to the target material, and no degeneration occurs even during thermal/chemical treatment. In addition, the small size of the molecule can be used as a fusion protein by attaching to other proteins. Specifically, since it can be used by attaching to a polymer protein chain, it can be used as a diagnostic kit and drug delivery material.

As used herein, the term “aptamer” refers to a kind of polynucleotide consisting of a particular single-stranded nucleic acid (DNA, RNA or modified nucleic acid) which has a stable tertiary structure and is capable of binding with high affinity and specificity to a target molecule. As described above, aptamers are composed of polynucleotides that can bind specifically to antigenic substances like antibodies, but are more stable than proteins, has simple structure, and easy to synthesize, and thus it can be used in place of an antibody.

On the other hand, the kit for diagnosing SLE or predicting SLE prognosis may further comprise one or more other component, compositions, solutions or devices suitable for analytical methods.

In addition, the present invention provides a method of providing information necessary for predicting SLE prognosis comprising: (1) measuring AIMP1 level from samples isolated from SLE patients; and (2) determining as active SLE when a measured AIMP1 level is 10 to 20 ng/mL.

As used herein, the term “sample” includes samples such as tissue, cells, blood, serum, plasma, saliva, sputum, cerebrospinal fluid, or urine that differs from the control in AIMP1 levels, but it is not limited thereto. Preferably it may be blood, more preferably serum.

Specifically, the method of measuring the AIMP1 level may be specifically, using an antibody that specifically binds to the AIMP1, more specifically, immunoassay, ligand binding assay, MALDI-TOF (Matrix Desorption/Ionization Time of Flight Mass Spectrometry), SELDI-TOF (Surface Enhanced Laser Desorption/Ionization Time of Flight Mass Spectrometry), radioimmunoassay, radial immunodiffusion assay, Ouchterlony immunodiffusion assay, rocket immunoelectrophoresis, complement fixation assay, two-dimensional electrophoresis analysis, liquid chromatography-mass spectrometry (LC-MS), Liquid chromatography-mass spectrometry/mass spectrometry (LC-MS/MS) or enzyme linked immunosorbent assay (ELISA), but it is not limited thereto.

Hereinafter, examples will be described in detail to understand the present invention. However, the following examples are merely to illustrate the content of the present invention is not limited to the scope of the present invention. The examples of the present invention are provided to more completely explain the present invention to those skilled in the art.

### EXPERIMENTAL EXAMPLE

The following Experimental Examples are intended to provide experimental examples that are commonly applied to each example according to the present invention.

1. Patients

The present inventors reviewed the medical records of 160 patients with SLE, who had been first diagnosed with SLE at the Division of Rheumatology, Yonsei University College of Medicine, Severance Hospital and who provided their blood for serum storage from March 2015 to September 2016. The inclusion criteria were as follows:

1) patients who fulfilled the 1997 revised American College of Rheumatology classification criteria for SLE; 2) those who had no medical conditions influencing serum AIM P1, erythrocyte sedimentation rate (ESR) and C-reactive protein (CRP) such as malignancies, infectious diseases and autoimmune diseases other than SLE; 3) those who had well-described medical documents regarding both clinical and laboratory items of SLEDAI-2K assessed and measured on the same day of serum storage; 4) those who had laboratory results related to inflammatory burdens other than SLEDA1-2K measured on the same day of serum storage. Serum samples of healthy control (n=43) were obtained from healthy volunteers after informed consents were provided at the Severance Hospital Health Centre. This study was approved by the Institutional Review Board of Severance Hospital and conducted in accordance with the principles set forth in the Declaration of Helsinki.

2. Clinical and Laboratory Data, Medications

Demographic data included age, gender and disease duration. SLEDAI-2K was used as an index for disease activity of SLE and it was calculated using clinical features and collecting laboratory results belonging to SLEDAI-2K such as anti-ds DNA, complement (C)3, C4, and counts of white blood cells (WBCs), lymphocytes and platelets, and haemoglobin on the same day as serum storage. Also the present inventors reviewed laboratory data other than laboratory items of SLEDAI-2K reflecting the inflammatory burdens of SLE such as ESR and CRP. The present inventors set the cut-off of SLEDAI-2K at 5 to divide active and stable SLE, and active SLE was defined when patients had a sum of SLEDAI-2K scores more than 5. All laboratory data obtained was estimated on the same date of serum storage. Medications were identified using the Korean Drug Utilization Review system, and only medications that were currently being administered was counted.

3. Measurement of Serum AIMP1

The present inventors measured serum AIMP1 level using stored serum samples of SLE patients and healthy controls. Human AIMP1 ELISA kits were purchased from Cloud-Clone Corp. (Houston, Tex. 77084, USA), and AIMP1 levels were measured according to the manufacturer's instructions. Briefly, sample was diluted with PBS at a ratio of 1:5, and 100 ml sample was added to each well, covered with the plate sealer and incubated for 1 h at 37° C. Then, 100 ml of detection reagent A working solution to each well was added and covered with the plate sealer and incubated for 1 h at 37° C. Each well was washed 3 times with 350 ml of washing solution. 100 ml of detection reagent B working solution was added to each well, covered with plate sealer and incubated for 30 min at 37° C. Plate was washed 5 times with washing buffer. 100 ml of 3,3′,5,5′-tetramethylbenzidine (TMB), substrate solution, was added and incubated 15 min at room temperature without light. Then, 50 ml of Stop solution (0.1 N Sulfuric acid) was added and O.D value of each well was measured at 450 nm.

4. Statistical Analysis

Continuous variables were presented as median with inter-quartile ranges (IQR), and categorical variables were expressed as frequencies and percentages. Continuous variables were compared using the Student's t-test, and categorical data were compared using the chi-square test or Fisher's exact test. Correlations between serum AIMP1 with SLEDAI-2K and laboratory variables related to either disease activity or inflammatory burdens were evaluated by using the Pearson's correlation analysis. The odds ratio (OR) was assessed using multivariate logistic regression for all variables with p-values <0.05 in univariate analysis. The optimal cut-off value of serum AIMP1 to predict active SLE was evaluated by calculating the area under the receiver operator characteristic curve (AUROC), and the relative risk (RR) of serum AIMP1 for active and stable SLE was analysed using contingency tables and the chi-square test. All statistical analyses were conducted using both GraphPad Prism version 5.0 (GraphPad Software, San Diego, Calif., USA) and the SPSS package for Windows version 21 (SPSS Inc., Chicago, Ill., USA), and a two-tailed p-value <0.05 was considered statistically significant.

### <Example 1> Characteristics of Patients with Active and Stable SLE

The characteristics of SLE patients are shown in Table 1. The median age was 41.0 and 90.0% of patients were female. The median disease duration of subjects was 79.0 months. The median SLEDAI-2K and serum AIMP1 level were 4.5 and 6.8 ng/mL, respectively. Glucocorticoid was the most commonly administered medication (76.8%) followed by hydroxychloroquine (42.5%) and mycophenolate mofetil (22.5%).

All SLE patients were evenly reclassified as active and stable SLE groups according to the cut-off of SLEDAI of 5 (80 patients for each group). There were no significant differences in age and gender between the two groups, while, patients with stable SLE had longer disease durations than those with active SLE. Patients with active SLE had the higher median SLEDAI-2K than those with stable SLE (7.0 vs. 2.0, p<0.001). In addition, patients with active SLE showed laboratory results closely correlated with increased disease activity of SLE, except white blood cell counts, and those reflecting extended inflammatory burdens. Patients with active SLE had the higher median serum AIMP1 than those with stable SLE (8.0 vs. 6.4 ng/mL, p<0.001). Difference in medications concurrently administered was not statistically apparent between patients with active and stable SLE (Table 1).

TABLE 1

| | | | |
|---|---|---|---|
| Total | Active SLE | Stable SLE | |
| (n = 160) | (n = 80) | (n = 80) | p-value |

Demographic data

| | | | | | | | |
|---|---|---|---|---|---|---|---|
| Age (years) | 41.0 | (19.5) | 40.0 | (20.0) | 42.5 | (19.0) | 0.071 |
| Female gender (N, (%)) | 144 | (90.0) | 74 | (92.5) | 70 | (87.5) | 0.293 |
| Disease duration (months) | 790 | (146.5) | 63.5 | (118.5) | 101.0 | (162.5) | 0.003 |
| SLEDAI-2K | 4.5 | (5.0) | 7.0 | (3.5) | 2.0 | (4.0) | <0.001 |

Laboratory variables related to disease activity and inflammatory burdens

| | | | | | | | |
|---|---|---|---|---|---|---|---|
| Anti-ds DNA (IU/mL) | 17.0 | (83.5) | 58.0 | (207.0) | 0.0 | (21.0) | <0.001 |
| Complement 3 (mg/dL) | 73.2 | (43.0) | 64.8 | (32.1) | 90.9 | (38.5) | <0.001 |
| Complement 4 (mg/dL) | 13.9 | (13.1) | 9.4 | (9.4) | 18.3 | (8.2) | <0.001 |
| White blood cell count (/μL) | 5290.0 | (3315.0) | 4995.0 | (3720.0) | 5625.0 | (3205.0) | 0.491 |
| Lymphocyte count (/μL) | 1210.0 | (1010.0) | 885.0 | (705.0) | 1615.0 | (950.0) | <0.001 |
| Haeoglobin (g/dL) | 12.1 | (2.3) | 11.3 | (3.1) | 12.9 | (1.8) | <0.001 |
| Platelet count (×1,000/μL) | 205.0 | (104.5) | 180.0 | (121.0) | 234.0 | (88.0) | <0.001 |
| ESR (mm/hr) | 28.5 | (30.0) | 37.0 | (43.5) | 24.0 | (19.5) | <0.001 |
| CRP (mg/L) | 1.2 | (2.8) | 1.6 | (7.1) | 0.7 | (1.9) | 0.002 |
| Serum AIMP1 (ng/mL) | 6.8 | (4.8) | 8.0 | (7.7) | 6.4 | (3.7) | <0.001 |

Medications (N, (%))

| | | | | | | | |
|---|---|---|---|---|---|---|---|
| Glucocorticoid | 123 | (76.8) | 63 | (78.7) | 60 | (75.0) | 0.575 |
| Hydroxychloroquine | 68 | (42.5) | 29 | (36.2) | 39 | (48.7) | 0.110 |
| Cyclophosphamide | 3 | (1.8) | 3 | (3.7) | 0 | (0.0) | 0.245 |
| Mycophenolate mofetil | 36 | (22.5) | 15 | (18.7) | 21 | (26.2) | 0.257 |
| Tacrolimus | 9 | (5.6) | 5 | (6.2) | 4 | (5.0) | 0.999 |
| Azathioprine | 14 | (8.7) | 8 | (10.0) | 6 | (7.5) | 0.577 |

Values are expressed as the median (interquartile range) or n (%).
SLEDAI-2K, Systemic lupus erythematosus disease activity index-2000; AIMP1, Aminoacyl tRNA synthetase complex interacting multifunctional protein 1; ESR Erythrocyte sedimentation rate; CRP, C-reactive protein; BUN, Blood urea nitrogen; Cr, Creatinine; AST, Aspartate aminotransferase; ALT, Alanine aminotransferase.

### <Example 2> Comparison of Serum AIMP1 Between Patients with SLE and Healthy Controls

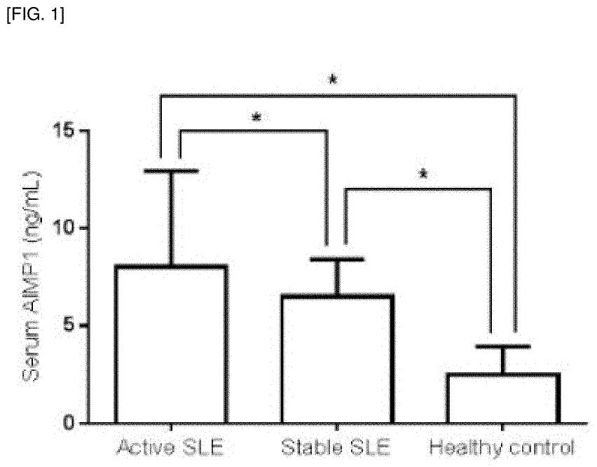

When serum AIMP1 between patients with SLE and healthy controls was compared, it was found that both patients with active and stable SLE showed the higher median serum AIMP1 than healthy controls (the median serum AIMP1 of active SLE vs. the median of healthy controls, p<0.001, and the median serum AIMP1 of stable SLE vs. the median of healthy controls, p<0.001) (FIG. 1).

### <Example 3> Correlation of Serum AIMP1 with Laboratory Variables Related to Either Disease Activity or Inflammatory Burdens in Patients with SLE

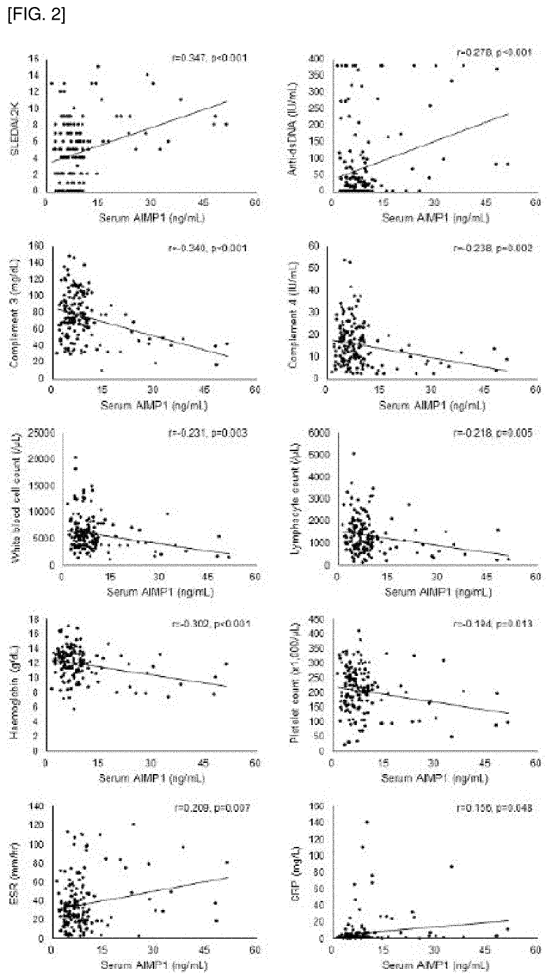

The present inventors evaluated the correlation of serum AIMP1 with SLEDAI-2K and laboratory variables related to either disease activity or inflammatory burdens in patients with SLE. Serum AIMP1 was significantly correlated with SLEDAI-2K (r=−0.347, p<0.001) and furthermore, serum AIMP1 was also correlated with laboratory variables related to either disease activity or inflammatory burdens. Among laboratory variables, serum AIMP1 was the most strongly correlated with C3 (r=−0.340, p<0.001) and subsequently with haemoglobin (r=−0.302, p<0.001) and anti-ds DNA (r=0.278, p<0.001) (FIG. 2).

### <Example 4> Serum AIMP1 is a Useful Predictive Marker for Active SLE

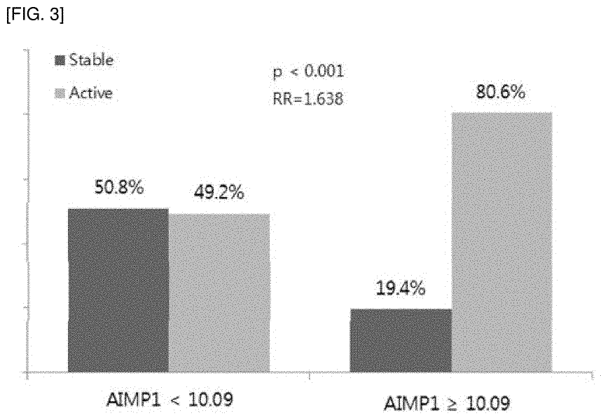

The present inventors calculated the optimal cut-off of serum AIMP1 to predict active SLE by using ROC analysis. The optimal cut-off to predict active SLE of serum AIMP1 was found to be 10.09 ng/mL (AUROC 0.634, 95% confidence interval (CI) 0.554-0.708, p=0.002). By classifying patients into two groups according to the optimal cut-off, active SLE in patients with serum AIMP1≥10.09 ng/mL was observed more frequently than in those without (80.5% vs. 49.1%, p<0.001). Moreover, the risk of active SLE in patients with serum AIMP1≥10.09 ng/mL was much higher than that in patients without (RR 1.638, 95% CI 1.287-2.082, p<0.001) (FIG. 3).

Finally, univariate and multivariate logistic regression analysis was performed to clarify the potential of serum AIMP1 to predict active SLE based on a SLEDAI-2K. In univariate analysis, laboratory variables related to either disease activity or inflammatory burdens except white blood cell count were shown to be useful in discriminating active and stable SLE. However, in multivariate analysis, serum AIMP1≥10.09 ng/mL (odds ratio (OR) (3.919, 95% CI 1.222-12.564, p=0.021), C3 (OR 0.957, 95% CI 0.938-0.976, p<0.001), lymphocyte count (OR 0.998, 95% CI 0.997-0.999, p<0.001), ESR (OR 1.029, 95% CI 1.007-1.051, p=0.008) revealed to be useful in discriminating between active and stable SLE (Table 2).

TABLE 2

| | Univariate analysis | | | Multivariate analysis | | |
|---|---|---|---|---|---|---|
| | Odds | | | Odds | | |
| Laboratory variables | ratio | 95% CI | p-value | ratio | 95% CI | p-value |
| Serum AIMP1 ≥ 10.09 (ng/mL) | 5.930 | 2.412-14.579 | <0.001 | 3.919 | 1.222-12.564 | 0.021 |
| Anti-ds DNA (IU/mL) | 1.013 | 1.006-1.019 | <0.001 | | | |
| Complement 3 (mg/dL) | 0.945 | 0.928-0.962 | <0.001 | 0.957 | 0.938-0.976 | <0.001 |
| Complement 4 (mg/dL) | 0.889 | 0.849-0.931 | <0.001 | | | |
| White blood cell count (/μL) | 1.000 | 0.999-1.000 | 0.489 | | | |
| Lymphocyte count (/μL) | 0.998 | 0.997-0.998 | <0.001 | 0.998 | 0.997-0.999 | <0.001 |
| Haemoglobin (g/dL) | 0.576 | 0.462-0.720 | <0.001 | | | |
| Platelet count (×1,000/μL) | 0.990 | 0.985-0.995 | <0.001 | | | |
| ESR (mm/hr) | 1.036 | 1.019-1.053 | <0.001 | 1.029 | 1.007-1.051 | 0.008 |
| CRP (mg/L) | 1.066 | 1.010-1.126 | 0.019 | | | |

AIMP1, Aminoacyl tRNA synthetase complex interacting multifunctional protein 1; ESR, Erythrocyte sedimentation rate; CRP, C-reactive protein.

While the present invention has been particularly described with reference to specific embodiments thereof, it is apparent that this specific description is only a preferred embodiment and that the scope of the present invention is not limited thereby to those skilled in the art. That is, the practical scope of the present invention is defined by the appended claims and their equivalents.

1-8. (canceled)

9. A composition for predicting SLE prognosis comprising an agent capable of measuring level of AIMP1 as an active ingredient.

10. The composition for predicting SLE prognosis of claim 9, wherein the agent capable of measuring the level of AIMP1 is antibody, peptide, aptamer or compound, which specifically binds to the AIMP1.

11. A kit for predicting SLE prognosis comprising the composition of claim 9.

12. A method of providing information necessary for predicting SLE prognosis comprising:
(1) measuring AIMP1 level from samples isolated from SLE patients; and
(2) determining as active SLE when a measured AIMP1 level is 10 to 20 ng/mL.

13. The method of providing information necessary for predicting SLE prognosis of claim 12, wherein the sample is blood.

14. A method for diagnosing and treating systemic lupus erythematosus (SLE) in a subject, the method comprising:
a) obtaining a sample from the subject;
b) detecting whether AIMP1 is present in the sample;
c) diagnosing the subject as having active SLE when the measured AIMP1 level is 10-20 ng/ml; and
d) administering an effective SLE treatment to the subject diagnosed as having an active SLE.

15. The method of claim 14, wherein the sample is blood.

16. The method of claim 14, wherein the b) detecting is carried out using one or more method selected from the group consisting of:
(i) an immunoassay using an antibody capable of specifically binding to the AIMP1,
(ii) a ligand binding assay using a ligand capable of binding to the AIMP1,
(iii) MALDI-TOF (Matrix Desorption/Ionization Time of Flight Mass Spectrometry),
(iv) SELDI-TOF (Surface Enhanced Laser Desorption/Ionization Time of Flight Mass Spectrometry),
(v) radioimmunoassay,
(vi) radial immunodiffusion assay,
(vii) Ouchterlony immunodiffusion assay,
(viii) rocket immunoelectrophoresis,
(ix) complement fixation assay,
(x) two-dimensional electrophoresis analysis,
(xi) liquid chromatography-mass spectrometry (LC-MS),
(xii) Liquid chromatography-mass spectrometry/mass spectrometry (LC-MS/MS), and
(xiii) enzyme linked immunosorbent assay (ELISA).
